(19) United States
(12) Patent Application Publication
(10) Pub. No.: US 2025/0368835 A1
(43) Pub. Date: Dec. 4, 2025

(54) ANTIBACTERIAL AGENT AND ANTIVIRAL AGENT

(71) Applicant: DIC Corporation, Tokyo (JP)

(72) Inventors: Yasuhiro Kuwana, Kamisu-shi (JP); Momoka Kusano, Kamisu-shi (JP)

(73) Assignee: DIC Corporation, Tokyo (JP)

(21) Appl. No.: 19/206,508

(22) Filed: May 13, 2025

(30) Foreign Application Priority Data
May 28, 2024 (JP) 2024-086313

(51) Int. Cl. C09D 5/14; C09D 7/41; C09D 7/61

(52) U.S. Cl. CPC C09D 5/14; C09D 7/41; C09D 7/61

(57) ABSTRACT

Provided is an antibacterial and antiviral agent with relatively little change in hue and improved productivity, the agent being organic chromatic or organic achromatic and relatively less hazardous to human health. Inks, printed matter, paints, coatings, plastics, fibers, films, cosmetics, and the like containing the antibacterial and antiviral agent are also provided. It has been found that a composition including an organic pigment and a copper compound carried thereon acts as an antibacterial and antiviral agent, and products containing the composition also have antibacterial or antiviral activity similarly. This finding has led to achievement of the above-described object.

### BACKGROUND OF THE INVENTION

### 1. Field of the Invention

The present invention relates to an antibacterial and antiviral agent.

### 2. Description of the Related Art

In recent years, there has been a strong demand to maintain the cleanliness of living spaces, and air purifiers, disinfectant sprays, and the like are often installed in homes and public rooms to maintain clean environments. Air purifiers and disinfectant sprays are now serving an antibacterial function, an antiviral function, an allergen substance-removing function, and a deodorizing function to provide additional values, beyond a mere air purification function of removing dirt and dust in the air. Applications requiring such additional values include, for example, textile, plastic, paint, and ink applications.

In textile applications, antibacterial activity or antiviral activity can be imparted by kneading an antibacterial or antiviral agent into fibers or by fixing a solution containing an antibacterial or antiviral agent to fiber surfaces. In the case of kneading into fibers, although wash durability is generally considered to be high, there is concern that an organic antibacterial or antiviral agent may thermally decompose due to high temperatures during spinning, and improvement in heat resistance has been desired. On the other hand, in the case of fixing to fiber surfaces, although an organic antibacterial or antiviral agent, such as a quaternary ammonium salt, is generally used, the antibacterial or antiviral activity deteriorates with washing (Japanese Unexamined Patent Application Publication No. 2013-76188).

In paint applications, an organic antibacterial or antiviral agent is used in the same manner as in textile applications. However, for example, acrylic/melamine-based paints require a curing process by heat treatment, and an organic antibacterial or antiviral agent, which is considered to have low heat resistance, may cause thermal degradation of a surface to be treated and an object to be treated (Japanese Unexamined Patent Application Publication No. 2017-014401). From this perspective, improvement of heat resistance has been desired in applications that involve treatment at high temperatures.

In terms of the amount of antibacterial or antiviral agent contained, all applications require that the effect is expressed with a relatively small amount, and the key technical issue is how to efficiently expose the antibacterial or antiviral agent to the surface of fibers, plastics, coating films, or the like (Japanese Unexamined Patent Application Publication No. 2006-28453 and Japanese Unexamined Patent Application Publication No. 2022-93225).

In the textile, plastic, paint, and ink applications, coloring agents are usually contained. From the perspective described above, there is an example of antibacterial compositions in which antibacterial activity is imparted by sputtering a metal compound on an organic pigment and coating the organic pigment surface with the metal compound (Japanese Unexamined Patent Application Publication No. H8-239302). However, the sputtering on powder such as organic pigment has very low productivity, because the quantity that can be processed at a time is small, and the sputtering takes time. In addition, the antibacterial composition has a problem in that the coloration of the organic pigment is significantly impaired because the entire surface of the pigment particle is covered with the metal compound.

In inorganic pigments, there is an example of antibacterial compositions in which antibacterial activity is imparted by carrying a silver oxide on the surface of an inorganic pigment (Japanese Unexamined Patent Application Publication (Translation of PCT Application) No. 2006-523735). The silver oxide itself is usually black to brown in color, and the silver oxide is formed in nanoparticles to the extent that no change in hue is observed. However, silver-based nanoparticles can enter and destroy cells in the human body. Even a small amount of silver oxide nanoparticles having fallen out can enter the human body through the skin and may adversely affect health.

An object of the present invention is therefore to provide an organic chromatic or organic achromatic antibacterial and antiviral composition with relatively little change in hue and improved productivity. In addition, the antibacterial and antiviral composition is relatively less hazardous to human health.

### SUMMARY OF THE INVENTION

An object of the present invention is to provide an antibacterial and antiviral agent with relatively little change in hue and improved productivity, the agent being organic chromatic or organic achromatic and relatively less hazardous to human health. Inks, printed matter, paints, coatings, plastics, fibers, films, cosmetics, and the like containing the antibacterial and antiviral agent are also provided.

The inventors of the present invention have conducted elaborate studies to achieve the above object and found that a pigment composition including an organic pigment and a copper compound carried thereon acts as an antibacterial and antiviral agent, and products containing the composition also have antibacterial or antiviral activity similarly. This finding has led to achievement of the above-described object.

More specifically, the present invention includes the following.
[1] An antibacterial and antiviral agent containing a pigment composition in which a copper compound is carried on an organic pigment or dye.
[2] The antibacterial and antiviral agent according to [1], wherein a mass ratio between the organic pigment or dye and the copper compound in the pigment composition is organic pigment:copper compound=99.9:0.1 to 70:30.
[3] The antibacterial and antiviral agent according to [1] or [2], wherein the organic pigment is at least one or more compounds selected from phthalocyanine compounds, quinacridone compounds, and isoindolinone compounds.
[4] The antibacterial and antiviral agent according to [1] or [2], wherein the copper compound is carried in the form of at least one or more compounds selected from Cu₂Cl(OH)₃, CuO, Cu₂O, and CuCl₂·3Cu(OH)₂.
[5] An ink, printed matter, paint, coating, plastic, fiber, film, and cosmetic containing the antibacterial and antiviral agent according to any one of [1] to [4].

### DETAILED DESCRIPTION OF EMBODIMENTS

The following embodiments of the present invention are only some of the embodiments of the present invention and are not limited only to the contents of the description, provided that they do not substantially deviate from the spirit of the invention.

An antibacterial and antiviral agent according to the present invention will be described below.

### Organic Pigment

Examples of an organic pigment to be used in the present invention include azo, phthalocyanine, anthraquinone, perylene, perinone, quinacridone, thioindigo, dioxazine, isoindolinone, quinophthalone, azomethine, diketopyrrolopyrrole, isoindoline, lactam, and bipyrrolinone pigments. Examples include Red No. 3, Red No. 104, Red No. 106, Red No. 201, Red No. 202, Red No. 204, Red No. 205, Red No. 220, Red No. 223, Red No. 226, Red No. 227, Red No. 228, Red No. 230, Red No. 401, Red No. 405, Red No. 505, Orange No. 203, Orange No. 204, Orange No. 205, Yellow No. 4, Yellow No. 5, Yellow No. 401, Blue No. 1, Blue No. 404, C.I. Pigment Red 1, 2, 3, 4, 5, 6, 7, 8, 9, 12, 14, 15, 16, 17, 21, 22, 23, 31, 32, 37, 38, 41, 47, 48, 48:1, 48:2, 48:3, 48:4, 49, 49:1, 49:2, 50:1, 52:1, 52:2, 53, 53:1, 53:2, 53:3, 57, 57:1, 57:2, 58:4, 60, 63, 63:1, 63:2, 64, 64:1, 68, 69, 81, 81:1, 81:2, 81:3, 81:4, 83, 88, 90:1, 101, 101:1, 104, 108, 108:1, 109, 112, 113, 114, 122, 123, 144, 146, 147, 149, 151, 166, 168, 169, 170, 172, 173, 174, 175, 176, 177, 178, 179, 181, 184, 185, 187, 188, 190, 193, 194, 200, 202, 206, 207, 208, 209, 210, 214, 216, 220, 221, 224, 230, 231, 232, 233, 235, 236, 237, 238, 239, 242, 243, 245, 247, 249, 250, 251, 253, 254, 255, 256, 257, 258, 259, 260, 262, 263, 264, 265, 266, 267, 268, 269, 270, 271, 272, 273, 274, 275, 276, 277, 278, 279, 280, 281, 282, 283, 284, 285, 286, 287, 291, 295, 296, C.I. Pigment Blue 1, 1:2, 9, 14, 15, 15:1, 15:2, 15:3, 15:4, 15:6, 16, 17, 19, 25, 27, 28, 29, 33, 35, 36, 56, 56:1, 60, 61, 61:1, 62, 63, 66, 67, 68, 71, 72, 73, 74, 75, 76, 78, 79, C.I. Pigment Yellow 1, 2, 3, 4, 5, 10, 12, 13, 14, 15, 16, 17, 18, 24, 31, 32, 34, 35, 35:1, 36, 36:1, 37, 37:1, 40, 42, 43, 53, 55, 60, 61, 62, 63, 65, 73, 74, 77, 81, 83, 93, 94, 95, 97, 98, 100, 101, 104, 106, 108, 109, 110, 113, 114, 115, 116, 117, 118, 119, 120, 123, 126, 127, 128, 129, 138, 139, 147, 148, 150, 151, 152, 153, 154, 155, 156, 166, 167, 168, 169, 170, 171, 172, 173, 174, 175, 176, 177, 179, 180, 181, 182, 185, 187, 188, 192, 193, 194, 196, 198, 199, 213, 214, C.I. Pigment Violet 1, 1:1, 2, 2:2, 3, 3:1, 3:3, 5, 5:1, 14, 15, 16, 19, 23, 25, 27, 29, 31, 32, 37, 39, 42, 44, 47, 49, 50, C.I. Pigment Green 1, 2, 4, 7, 8, 10, 13, 14, 15, 17, 18, 19, 26, 36, 37, 45, 48, 50, 51, 54, 55, 58, 59, C.I. Pigment Black 1, 7, 31, 32. These organic pigments may be used alone or in combination of two or more as appropriate depending on the required hue.

Natural colorant may be used as a dye to be used in the present invention. Examples of the natural colorant include carotenoids, anthocyanins, flavonoids, quinones, porphyrins, diketones, betacyanins, and azaphilones. For example, beta-carotene, norbixin, bixin, capsanthin, lutein, lycopene, crocin, crocetin, astaxanthin, cyanidin acyl glucoside, cyanidin, aglycone of peonidin, cyanidin glucoside, delphinidin glucoside, anthocyanin, shisonin, malonylshisonin, pelargonidin acyl glucoside, cocoa polyphenols, apigenidin, luteolinidin, polymerized proanthocyanidins, safflomin, carthamin, carminic acid, laccaic acid, chlorophyll, phycocyanin, curcumin, betanin, isobetanin, ankaflavin, monascorubrin, iridoid glycosides, ester hydrolysates of iridoid glycosides, and eumelanin may be used. These natural colorants may be used alone or in combination of two or more as appropriate depending on the desired hue.

### Phthalocyanine

In phthalocyanine, which is one of the organic pigments to be used in the composition according to the present invention, there is no restriction in metal-free phthalocyanine or central metal, but specific examples include sodium, magnesium, aluminum, silicon, potassium, calcium, titanium, vanadium, manganese, iron, cobalt, nickel, copper, gallium, germanium, zirconium, cadmium, indium, and tin. These metals and the phthalocyanine ring may be unsubstituted or may have a substituent such as halogen, carboxylic acid, hydroxyl group, or carbonyl group. In theory, the number of substituents (n) can be n=0 to 16, but in order to express the function as a pigment, a substituent(s) can be included to such a degree not soluble in water or a solvent.

In particular, iron phthalocyanine whose central metal is iron, copper phthalocyanine whose central metal is copper, cobalt phthalocyanine whose central metal is cobalt, and aluminum phthalocyanine whose central metal is aluminum have high antibacterial or antiviral effect and are suitable in the pigment composition according to the present invention.

The phthalocyanine to be used in the present invention is represented by the following general formula.

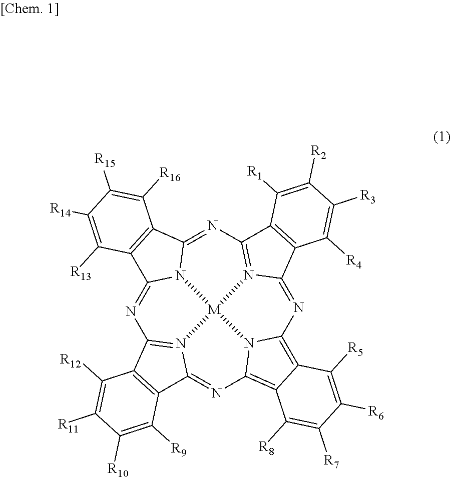

where M represents a metal of Na2, Mg, Al, Si, K2, Ca, Ti, V, Mn, Fe, Co, Ni, Cu, Ga, Ge, Zr, Cd, or Sn, or an oxy metal or a halogenated metal thereof; and R1 to R16 each independently represent a hydrogen atom or a halogen atom.

When pigmented, the phthalocyanine to be used in the present invention expresses crystalline properties rather than molecular properties. Specifically, because of the crystalline properties, the antibacterial or antiviral effect extends not only to an area in contact with the phthalocyanine but also to an area slightly distant from the phthalocyanine. When it comes to the antibacterial or antiviral effect, in the case of dyes, the antibacterial activity disappears when the dye is covered with the resin of a coating film or the like, whereas in the case of pigments, the antibacterial or antiviral effect can be maintained even when the pigment is slightly covered with the resin of a coating film or the like.

The phthalocyanine may be in a particle or needle form. The particle size and the aspect ratio are not limited, but generally the phthalocyanine formed in microparticles and nanoparticles preferably has a particle size of 20 to 200 nm and an aspect ratio of about 1 to 5.

When the phthalocyanine is a dye, a redox reaction occurs, that is, a molecule transitions from HOMO to LUMO, or a specific functional group of bacteria or the like comes into contact with a specific atom, whereby a decomposition reaction proceeds.

On the other hand, when the phthalocyanine is water-insoluble, a band gap is produced by having a certain crystal shape, and electron exchange for progressing a reaction occurs through valence band and conduction band. When there are multiple pigments, electron transfer necessary for a reaction, such as hopping conduction, can take place between the pigments even in the presence of a resin. This enables electron injection into a specific functional group of bacteria or the like. Presumably, a copper compound functions as an electron donor to exhibit photocatalytic activity.

From the above perspective, it is assumed that, among the metal species of the phthalocyanine, a metal species in which redox of the metal ion easily occurs in an electrochemical reaction has higher antibacterial or antiviral activity than a metal species in which redox of the phthalocyanine ring easily occurs.

### Quinacridone

Quinacridone, which is one of the organic pigments to be used in the composition according to the present invention, is preferably represented by the following general formula.

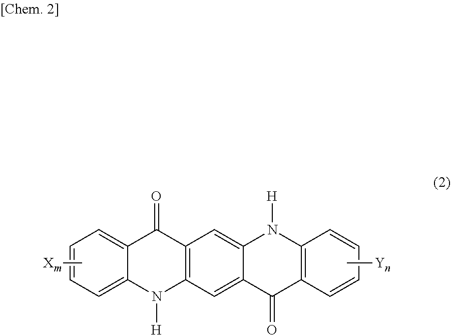

where X or Y each independently represent hydrogen, halogen, or an alkyl or alkoxy group having 1 to 4 carbon atoms; and m and n each independently represent an integer of 0 to 2.

Examples of the quinacridone represented by the general formula (2) include unsubstituted quinacridone pigments such as C.I. Pigment Violet 19, dimethylquinacridone pigments such as C.I. Pigment Red 122, and dichloroquinacridone pigments such as C.I. Pigment Red 202, C.I. Pigment Red 207, and C.I. Pigment Red 209.

Quinacridone pigments are known to have a certain crystal shape so that a band gap is produced and electron exchange for progressing a reaction occurs through valence band and conduction band, in the same manner as phthalocyanine pigments. When there are multiple pigments, electron transfer necessary for a reaction, such as hopping conduction, can take place between the pigments even in the presence of a resin. Thus, it is expected that a copper compound functions as an electron donor to exhibit photocatalytic activity to react with a specific functional group of bacteria or the like.

### Isoindolinone

Isoindolinone, which is one of the organic pigments to be used in the composition according to the present invention, is preferably represented by the following general formula.

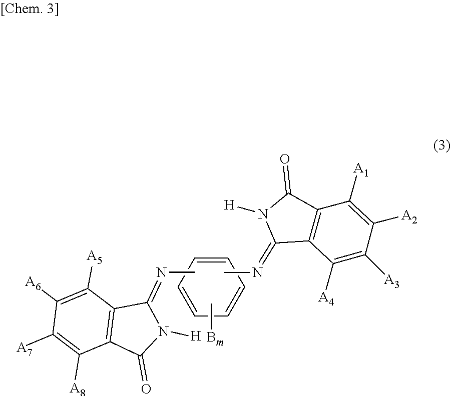

where A1 to A8 each independently represent hydrogen, halogen, or an alkyl or alkoxy group having 1 to 2 carbon atoms; B represents hydrogen, halogen, or an alkyl group having 1 to 2 carbon atoms; and m represents an integer of 1 to 3.

Examples of the isoindolinone represented by the general formula (2) include unsubstituted isoindolinone pigments such as C.I. Pigment Yellow 173, and chloro-substituted isoindolinone pigments such as C.I. Pigment Yellow 109 and C.I. Pigment Yellow 110.

Any known customary methods can be used for pigmentation of the organic pigment. Specifically, examples of the methods include: mixing and grinding a compound serving as the organic pigment with a water-soluble inorganic salt and a water-soluble organic solvent (solvent salt milling method); heating a compound serving as the organic pigment in a solvent in which the compound is insoluble (solvent method); and pulverizing using a pigment grinding machine or a pigment dispersing machine.

In the solvent salt milling method, for example, a compound serving as the organic pigment, a water-soluble inorganic salt such as sodium chloride or sodium sulfate, and a water-soluble organic solvent such as diethylene glycol or triethylene glycol are mixed and ground with heat, and washed with water.

When the solvent method is used, a liquid medium that does not dissolve a compound serving as the organic pigment is selected and used. As the liquid medium, a liquid medium that contains a water-soluble organic solvent as an essential component is preferably used in order to perform crystal control of the compound serving as the organic pigment more stably.

When the pulverizing method is used, for example, a pigment grinding machine or a pigment dispersing machine, such as a ball mill, a sand mill, an attritor, a horizontal continuous media disperser, a kneader, a continuous single-screw mixer, a continuous two-screw mixer, a three-roll mill, and an open-roll continuous mixer, can be used. The pigment grinding machine and the pigment dispersing machine can also be used in the solvent salt milling method.

### Copper Compound

A copper inorganic compound, a copper organic compound, or the like can be used as a starting material for a copper compound to be used in the present invention. Any copper compound can be used as the copper inorganic compound, such as copper(I) chloride, copper(II) chloride, copper(I) bromide, copper(II) bromide, copper sulfate, copper nitrate, copper iodate, copper perchlorate, copper phosphate, and copper pyrophosphate. These compounds may be used alone or in combination of two or more.

For example, copper formate, copper acetate, copper propionate, copper citrate, copper oxalate, copper ethoxide, copper isopropoxide, and copper butoxide can be used as the copper organic compound. These compounds may be used alone or in combination of two or more.

### Acidic Compound

An acidic compound to be used in the present invention is not essential in a method for producing the composition according to the present invention but preferably used because if used, a stable pigment composition can be obtained. Any known customary acidic compound can be used as the acidic compound and examples include hydrochloric acid, sulfuric acid, acetic acid, nitric acid, and phosphoric acid. These compounds may be used alone or in combination of two or more.

### Alkaline Compound

An alkaline compound to be used in the present invention is used to obtain the copper compound carried on the organic pigment in a method for producing the composition according to the present invention. Any known customary alkaline compound can be used as the alkaline compound and examples include sodium hydroxide, potassium hydroxide, tetramethylammonium hydroxide, tetrabutylammonium hydroxide, triethylamine, trimethylamine, ammonia, and basic surfactants. These compounds may be used alone or in combination of two or more.

### Pigment Composition

A pigment composition according to the present invention is a composition having antibacterial or antiviral activity, including a pigment composition in which the copper compound is carried on the organic pigment. The state of being carried means that the copper compound is scattered on the surface of a single particle of the organic pigment. The copper compound may be scattered uniformly or locally. The composition according to the present invention may also contain an organic pigment on which no copper compound is carried.

The copper compound carried on the organic pigment is mainly in the form of Cu₂Cl(OH)₃, CuO, Cu₂O, CuCl₂·3Cu(OH)₂ according to the analysis result of X-ray diffraction peaks and the result of X-ray fluorescence analysis. However, the compound may include a copper compound other than the above copper compounds, which is formed in the process of producing the pigment composition according to the present invention.

The carried copper compound has a particle size of about 1 to 100 nm, preferably 2 to 80 nm, and even more preferably 2 to 60 nm. The carried copper compound may have an irregular shape or may be needle-shaped, spherical, or polyhedral.

The ratio between the organic pigment and the copper compound in the pigment composition according to the present invention is organic pigment:copper compound=99.9:0.1 to 70:30 by mass, preferably 99.9:0.1 to 80:20, and particularly preferably 99.5:0.5 to 90:10. The above ratio between the organic pigment and the copper compound is preferred in terms of antibacterial or antiviral activity.

### Method for Producing Pigment Composition

A method for producing the pigment composition according to the present invention includes a step of mixing the organic pigment, the starting material, water, and an acidic compound, and an alkaline compound. For example, the organic pigment is first mixed and stirred in water, then the starting material, an aqueous solution of the starting material, or an acidic aqueous solution of the starting material with an acidic compound or an acidic aqueous solution of an aqueous solution of the starting material with an acidic compound is mixed and stirred. Subsequently, an alkaline compound or an aqueous solution of an alkaline compound is added under stirring or added dropwise. Alternatively, the organic pigment may be mixed and stirred in the acidic aqueous solution, then the starting material may be mixed and stirred, and an alkaline compound or an aqueous solution of an alkaline compound may be mixed and stirred.

In the method for producing the pigment composition according to the present invention, it is preferable that the starting material is completely dissolved before the addition of an alkaline compound so that it is carried on the entire organic pigment.

In the method for producing the pigment composition according to the present invention, a medium used for dispersing the organic pigment may be water alone or may contain an organic solvent if necessary. Examples of the organic solvent that can be used include alcohol solvents such as methanol, ethanol, 1-propanol, 2-propanol, and 1-butanol, ketone solvents such as acetone, methyl ethyl ketone, and methyl isobutyl ketone, dimethylformamide, N-methyl-2-pyrrolidone, and tetrahydrofuran. These organic solvents may be used alone or in combination of two or more kinds. When the organic solvent is not used, plasma treatment, corona treatment, high-pressure pulverization (dispersion) processing, bead pulverization (dispersion) processing, or the like can be performed on the organic pigment as it is or mixed with water in advance.

In the method for producing the pigment composition according to the present invention, the pH of the solution after mixing the alkaline compound is preferably in the range of pH 7 to 12 in order to produce the copper compound as described above, even more preferably pH 8 to 12, and particularly preferably pH 8 to 11.

This mixing step results in the copper compound carried on the organic pigment. The copper compound may be uniformly carried on the surface of the organic pigment or may be carried in a segregated state on a part of the surface of the organic pigment. The step after the mixing step is a step of separating solid and liquid, such as filtration, centrifugal separation, or sedimentation separation. The solid obtained in the separating step is, for example, washed, crushed, dried, and classified as necessary to obtain the composition according to the present invention.

The resulting pigment composition according to the present invention can be further heat treated. The heat treatment temperature is in the range of 150 to 400° C. and preferably in the range of 200 to 300° C. in order to prevent discoloration. Since it is said that Cu₂Cl(OH)₃ changes to CuO at 220° C. or higher, heat treatment at 220° C. or higher increases the ratio of CuO.

The ratio between the organic pigment and the copper compound in the pigment composition according to the present invention is organic pigment:copper compound=99.9:0.1 to 70:30 by mass, preferably 99.9:0.1 to 80:20, and particularly preferably 99:1 to 90:10. The above ratio between the organic pigment and the copper compound is preferred in terms of antibacterial or antiviral activity and hue.

The antibacterial and antiviral agent according to the present invention includes a pigment composition in which a copper compound is carried on an organic pigment or dye and includes other components other than the pigment composition, such as various inorganic pigments, various extender pigments, various natural extracts, various resins, and various additives, which are known and customary. The pigment composition and other components can be contained in any ratio in which the pigment composition is an essential component as long as antibacterial or antiviral activity is expressed. In terms of antibacterial or antiviral activity, the proportion of the pigment composition in the entire antibacterial and antiviral agent is preferably 20% by mass or more and particularly preferably 50% by mass or more.

In the present invention, “antibacterial” means encompassing the effect of reducing the number of bacteria, the effect of inactivating bacteria, and the effect of reducing the infectivity of bacteria. Similarly, in the present invention, “antiviral” means encompassing the effect of reducing the number of viruses, the effect of inactivating viruses, and the effect of reducing the infectivity of viruses.

In the present invention, the bacteria targeted for antibacterial action are not limited and can be any of bacteria and fungi. Examples of the bacteria include gram-negative bacteria such as E. coli, Pseudomonas aeruginosa, Salmonella, Moraxella, and Legionella; and gram-positive bacteria such as Staphylococcus aureus, Streptococcus pneumoniae, Bacillus cereus, Botulinum, and Clostridium bacteria. Examples of the fungi include yeasts such as Candida, rhodotorula, and baker's yeast; and molds such as red mold and black mold.

In the present invention, the viruses targeted for antiviral action are not limited and can be any of known enveloped viruses (viruses with envelopes) or non-enveloped viruses (viruses without envelopes).

Examples of the enveloped viruses include coronaviruses, influenza viruses, rubella viruses, Ebola viruses, measles viruses, varicella zoster viruses, herpes viruses, mumps viruses, arboviruses, RS viruses, SARS viruses, hepatitis viruses (e.g., hepatitis A virus, hepatitis B virus, hepatitis C virus, hepatitis D virus, hepatitis E virus), yellow fever viruses, AIDS viruses, rabies viruses, hantaviruses, dengue viruses, nipah virus, and Lyssavirus.

Examples of the non-enveloped viruses include adenovirus, norovirus, rotavirus, human papillomavirus, poliovirus, enterovirus, coxsackie virus, human parvovirus, encephalomyocarditis virus, polyomavirus, BK virus, rhinovirus, and feline calicivirus.

### Antibacterial Effect

Examples of indicators of the antibacterial effect include bacteria growth tests using a culturing kit and antibacterial activity tests defined in the JIS standards.

The main purpose of using a culturing kit is to grasp a phenomenon in which common bacteria and fungi existing in the natural world, such as in food, air, and water, grow in the culture medium of the culturing kit, by observing the development of colonies. When a substance having antibacterial effect is brought into contact with the culture medium, a certain amount of bacteria and fungi are killed, or their growth is inhibited, thereby preventing or delaying the development of colonies. The bacteria growth test using a culturing kit enables an evaluation by periodic observation of the above phenomenon.

Examples of the common bacteria and fungi include, but are not limited to, E. coli, Staphylococcus aureus, Bacillus cereus, Salmonella, Pseudomonas aeruginosa, fungi, and viable bacteria containing the above bacteria and fungi.

A common kit can be used as the culturing kit. Examples thereof include a simple microbial counting instrument San-ai Biochecker (manufactured by San-Ai Oil Co., Ltd.), a culture medium for bacteria counting Compact Dry (manufactured by Nissui Pharmaceutical Co., Ltd.), and the like.

The antibacterial activity tests defined in the JIS standards mainly target typical gram-negative bacteria such as E. coli, Pseudomonas aeruginosa, Klebsiella pneumoniae, and Moraxella, and gram-positive bacteria such as Staphylococcus aureus, MRSA, and Streptococcus pyogenes. In summary, a sample is inoculated with a test bacteria solution, the solution in contact with a film or glass is irradiated with light or kept in a dark place for a certain period of time, and then the collected bacteria solution is diluted and incubated on an agar medium. After incubation, the antibacterial activity value is determined by comparing the counts of colonies generated. Specifically, examples include JIS R1702 Test method for antibacterial activity of photocatalytic materials and efficacy, JIS R1752 Test method for antibacterial activity of photocatalytic materials and efficacy under indoor lighting environment, JIS L1902 Determination of antibacterial activity and efficacy of textile products, JIS Z2801 Antibacterial products—Test for antibacterial activity and efficacy, or the Absorption method, the Transfer method, the Printing method, and the Halo method defined in JIS L1902 Determination of antibacterial activity and efficacy of textile products.

### Antiviral Effect

Examples of indicators of the antiviral effect can include antiviral activity tests defined in the JIS standards and ISO standards.

The antiviral tests defined in the JIS standards and ISO standards mainly target typical influenza virus having envelope, feline calicivirus having no envelope, or bacteriophages. In summary, a sample is inoculated with a virus or bacteriophage solution, the solution in contact with a film or glass is irradiated with light or kept in a dark place for a certain period of time, and then the collected solution is diluted and incubated on an agar medium. After incubation, the antiviral activity value is determined by comparing the plaque counts. Alternatively, a sample is inoculated with a virus solution, the sample in contact with a film or glass through the virus solution is irradiated with light or kept in a dark place for a certain period of time, and then the virus solution on the sample is washed and collected. Then, the antiviral activity value is determined by comparing the virus infectivity titers. Specifically, examples include JIS R1706 Determination of antiviral activity of photocatalytic materials—Test method using bacteriophage Qβ, JIS R1756 Determination of antiviral activity of photocatalytic materials under indoor lighting environment—Test method using bacteriophage Qβ, ISO 21702 Measurement of antiviral activity on plastics and other non-porous surfaces, and JIS L1922 Determination of antiviral activity of textile products.

### Coating Film Antibacterial Activity Evaluation

Antibacterial testing against Staphylococcus aureus (NBRC 12732) and E. coli (NBRC 3972) was performed by the following method.

A 5 cm×5 cm coating film cleaned by UV irradiation was inoculated with 0.4 ml of a test bacteria solution. The bacteria solution kept in close contact with a film or glass was left in a dark place for 24 hours. The collected bacteria solution was then diluted and incubated on an agar medium. After incubation, the antibacterial activity value was determined by comparing the counts of colonies formed. The calculation formula is written as R=(Uₜ−U₀)−(Aₜ−U₀)=Uₜ−Aₜ (R: antibacterial activity value, U₀: the mean logarithmic value of the viable bacteria count immediately after inoculation in the untreated product, Uₜ: the mean logarithmic value of the viable bacteria count after 24 hours in the untreated product, Aₜ: the mean logarithmic value of the viable bacteria count after 24 hours in the treated product). The treated product is a coating film obtained by forming a film of a dispersion containing a test target, and the untreated product is a PET film.

As a rough standard of the antibacterial activity value, for example, JIS Z 2801:2021 Antibacterial products—Test for antibacterial activity and efficacy stipulates that the criterion for antibacterial effect is 2.0 or higher. An antibacterial activity value of 2.0 means that the treated product suppressed the growth of bacteria by 99% after the test, compared with the untreated product. However, this does not necessarily mean that no bacteria grow.

### Coating Film Antiviral Activity Evaluation

Antiviral testing against bacteriophage Qβ (NBRC 20012, host E. coli (NBRC 106373)) was performed by the following method.

A 5 cm×5 cm coating film was inoculated with a test phage solution. The phage solution kept in close contact with a film or glass was left for four hours in a dark place. The collected phage solution was then diluted and incubated on an agar medium. After incubation, the antiviral activity value was determined by comparing the counts of colonies formed. The calculation formula is written as V_{D}: antiviral activity value (in a dark place): [V_{D}=Log(B_{D})−Log(C_{D})] (D: dark place, B_{D}: the infectivity titer in an untreated product after being left for four hours in a dark place, C_{D}: the infectivity titer in a treated product after being left for four hours in a dark place). The treated product is a coating film obtained by forming a film of a dispersion containing a test target, and the untreated product is a PET film.

Here, for example, an antiviral activity value of 2.0 means that the treated product suppressed the phage growth by 99% after the test, compared with the untreated product. However, this does not necessarily mean that no phage grows.

The antibacterial and antiviral agent according to the present invention can be used to provide inks, printed matter, paints, coatings, plastics, fibers, films, cosmetics, and the like. The applications detailed below are examples, and the antibacterial and antiviral agent according to the present invention can be used for any purpose in the applications having antibacterial, antifungal, sterilizing, and antiviral activities.

The antibacterial and antiviral agent according to the present invention has a structure in which a nano-sized copper compound of a few nm to a few tens of nm is carried on a pigment and thus presumably has bioactivities other than the above antibacterial and antiviral activities, namely, deodorant, anti-allergen, and anti-biofilm activities. For example, bacteria, mold, and the like prone to grow and cause bad odors and sliminess in places that are in frequent contact with water, such as drains, sinks, and bathrooms, due to high humidity and temperature. The present invention has antibacterial activity and is therefore thought to prevent the growth of bacteria, mold, and the like and to prevent the formation of biofilms, which cause bad odors and sliminess.

### Ink Applications

The antibacterial and antiviral agent according to the present invention can provide a printing ink having antibacterial or antiviral effect. The printing ink can be prepared by mixing the antibacterial and antiviral agent according to the present invention with a variety of known customary binder resins, solvents, additives, and the like according to a conventionally known preparation method. Specifically, a liquid ink can be prepared by preparing a base ink for liquid inks with a high pigment concentration and using a variety of binders, solvents, additives, and the like.

The antibacterial and antiviral agent according to the present invention can be used to produce PU and NC inks having antibacterial or antiviral effect and is suitable as an organic composition for gravure and flexographic printing inks. The PU ink is made of a PU resin, a pigment, a solvent, and a variety of additives. The NC ink is made of an NC resin, a pigment, a solvent, and a variety of additives. The PU resin is not limited as long as it has a urethane structure in the backbone, and includes polyurethane, polyurethane polyurea, and the like. Examples of the respective solvents include: aromatic organic solvents such as toluene and xylene; ketone solvents such as methyl ethyl ketone, methyl isobutyl ketone, cyclohexanone, 2-heptanone, and 3-heptanone; ester solvents such as ethyl acetate, n-propyl acetate, isopropyl acetate, isobutyl acetate, propylene glycol monoethyl ether acetate, and propylene glycol monomethyl ether acetate; alcohol solvents such as methanol, ethanol, n-propanol, isopropanol, n-butanol, isobutanol, and t-butanol; (poly)alkylene glycol monoalkyl ether solvents such as propylene glycol monoethyl ether, propylene glycol monomethyl ether, ethylene glycol monomethyl ether, ethylene glycol monoethyl ether, ethylene glycol mono-n-propyl ether, ethylene glycol mono-i-propyl ether, diethylene glycol monomethyl ether, diethylene glycol monoethyl ether, diethylene glycol mono-n-propyl ether, and diethylene glycol mono-i-propyl ether; (poly)alkylene glycol monoalkyl ether acetate solvents such as ethylene glycol monomethyl ether acetate, ethylene glycol monoethyl ether acetate, diethylene glycol monomethyl ether acetate, and diethylene glycol monoethyl ether acetate; and other ether solvents such as diethylene glycol dimethyl ether and diethylene glycol diethyl ether. The solvents may be used alone or in combination of two or more kinds. Examples of a variety of additives that can be used include anionic, nonionic, cationic, and zwitterionic surfactants, rosins such as gum rosin, polymerized rosin, disproportionated rosin, hydrogenated rosin, maleated rosin, hardened rosin, and phthalic alkyd resin, pigment derivatives, dispersants, wetting agents, adhesion aids, leveling agents, antifoaming agents, antistatic agents, trapping agents, anti-blocking agents, and wax components.

When the antibacterial and antiviral agent according to the present invention is used as a printing ink, the printing ink containing the composition according to the present invention prepared as described above can be diluted in ethyl acetate, polyurethane-based varnish, or polyamide-based varnish and used. The printing ink can be prepared by a known customary method.

### Paint Applications

When the antibacterial and antiviral agent according to the present invention is used in a paint having antibacterial or antiviral effect, various resins including acrylic, melamine, epoxy, polyester, polyurethane, polyamide, and phenolic resins can be used as the paint.

Examples of a solvent used in the paint include aromatic solvents such as toluene, xylene, and methoxybenzene; acetate solvents such as ethyl acetate, butyl acetate, propylene glycol monomethyl ether acetate, and propylene glycol monoethyl ether acetate; propionate solvents such as ethoxyethyl propionate; alcohol solvents such as methanol, ethanol, propanol, n-butanol, and isobutanol; ether solvents such as butyl cellosolve, propylene glycol monomethyl ether, diethylene glycol ethyl ether, and diethylene glycol dimethyl ether; ketone solvents such as methyl ethyl ketone, methyl isobutyl ketone, and cyclohexanone; aliphatic hydrocarbon solvents such as hexane; nitrogen compound solvents such as N,N-dimethylformamide, γ-butyrolactam, N-methyl-2-pyrrolidone, aniline, and pyridine; lactone solvents such as γ-butyrolactone; carbamate esters such as a mixture of methyl carbamate and ethyl carbamate in 48:52; and water. As the solvent, water-soluble polar solvents such as propionates, alcohols, ethers, ketones, nitrogen compounds, lactones, and water are particularly suitable.

When the pigment additives and/or composition is dispersed or mixed in a liquid resin to form a paint resin composition, ordinary additives such as dispersants, fillers, paint aids, drying agents, plasticizers, and/or auxiliary pigments can be used. This is accomplished by preparing each individual component or preparing some of the components together and collecting all the components, or adding all the components at once, and then dispersing or mixing the components.

Examples of a dispersing machine for dispersing the antibacterial and antiviral agent containing the composition prepared according to the application as described above include, but are not limited to, dispersers, homo mixers, paint conditioners, Scandex, bead mills, attritors, ball mills, two-roll mills, three-roll mills, pressure kneaders, and other known dispersing machines. In dispersing the composition, a resin and a solvent are added and dispersed to achieve a viscosity that allows dispersion in these dispersing machines. After dispersion, the high-concentration paint base has a solid content of 5 to 20%, and this paint base is further mixed with a resin and a solvent to be used as a paint.

### Plastic Applications

The antibacterial and antiviral agent according to the present invention can also be used in plastic applications having antibacterial or antiviral effect. To obtain plastic molded products, thermoplastic resins (plastics) for thermal molding such as injection molding and press molding, for example, polyolefins such as polyethylene and polypropylene and polyvinyl chloride resins, are used. The antibacterial and antiviral agent according to the present invention can be kneaded into these resins by a conventionally known method and used.

### Cosmetics Applications

The antibacterial and antiviral agent according to the present invention can be used in cosmetics. The cosmetics that are used are not limited, and the composition according to the present invention can be used in various types of cosmetics.

The cosmetics may be any type of cosmetics as long as they can effectively express the functions. The cosmetics may be lotions, cream gels, sprays, and the like. Examples of the cosmetics include skin care cosmetics such as facial cleansers, make-up removers, lotions, serums, facial masks, protective emulsions, protective creams, whitening cosmetics, and UV-protective cosmetics; make-up cosmetics such as foundations, face powders, makeup bases, lipsticks, eye makeup, blushers, and nail enamels; hair care cosmetics such as shampoos, hair conditioners, hair treatments, hairdressing agents, permanent wave agents, hair dyes, and hair growth tonics; and body care cosmetics such as body-wash products, deodorant cosmetics, and bath products.

The antibacterial and antiviral agent according to the present invention for use in the above cosmetics can be set as appropriate according to the type of cosmetics. The content in the cosmetics is usually in the range of 0.1 to 99% by mass, and typically, the amount is preferably in the range of 0.1 to 10% by mass. On the other hand, in make-up cosmetics, the amount may be in the range of 5 to 80% by mass, in the range of 10 to 70% by mass, or in the range of 20 to 60% by mass. When the amount of the antibacterial and antiviral agent according to the present invention contained in the cosmetics is within the above range, the functions such as coloring can be effectively expressed and the functions required for the cosmetics can be maintained.

In addition to the antibacterial and antiviral agent according to the present invention, the cosmetics can contain acceptable cosmetic ingredients according to the type of cosmetics, such as carrier, pigment, oil, sterol, amino acids, moisturizer, powder, coloring agent, pH adjuster, fragrance, essential oil, cosmetic active ingredient, vitamin, essential fatty acid, sphingolipid, self-tanning agent, excipient, filler, emulsifier, antioxidant, surfactant, chelating agent, gelling agent, thickener, emollient, wetting agent, moisturizer, mineral, viscosity modifier, fluidity modifier, keratolytic agent, retinoid, hormonal compound, alpha hydroxy acid, alpha keto acid, anti-mycobacterial agent, antifungal agent, antibacterial agent, antiviral agent, analgesic, antiallergic agent, antihistamine, anti-inflammatory agent, anti-irritant, antitumor agent, immune system booster, immune system suppressor, anti-acne agent, anesthetic agent, disinfectant, insect repellent, skin cooling compound, skin protectant, skin penetration enhancer, exfoliant, lubricant, aromatic, dye, decolorant, hypopigmenting agent, preservative, stabilizer, drug, light stabilizer, and spherical powder.

The cosmetics can be produced by mixing the antibacterial and antiviral agent according to the present invention and other cosmetic ingredients.

The cosmetics containing the antibacterial and antiviral agent according to the present invention can be used in the same way as ordinary cosmetics, according to the type of cosmetics.

When the antibacterial and antiviral agent according to the present invention is used in paints or plastics, it is preferable that the antibacterial and antiviral agent according to the present invention is present in the vicinity of a paint or plastic surface. The adjustments can be made by adjusting the type of copper compound or by controlling the dispersion conditions, according to the type of resin used in the paint or plastic.

It has been found that the antibacterial and antiviral activities are particularly excellent when the antibacterial and antiviral agent according to the present invention contains a pigment composition in which a copper compound is carried in the form of nanoparticles on the surface of a pigment having the properties of organic semiconductors, such as phthalocyanine and quinacridone. The reason is not clear, but a possible reason is that when the pigment composition with the copper compound carried on the surface of the pigment having the properties of organic semiconductors or the copper compound not carried migrates to the vicinity of the surface of the coating film or plastic, the remaining components tend to migrate to the vicinity of the surface together.

### EXAMPLES

The present invention will be further detailed below with examples, but the present invention is not limited to these examples. “%” in the compositions of the following examples means “% by mass”.

### Antibacterial Test 1

With reference to JIS R 1752: 2020, antibacterial testing was performed by the following method on a coating film against Staphylococcus aureus (NBRC 12732) as a representative of gram-positive bacteria and E. coli (NBRC 3972) as a representative of gram-negative bacteria.

A 5×5 cm coating film cleaned by heating in a dry heat sterilizer was inoculated with 0.1 ml of a test bacteria solution. The bacteria solution kept in close contact with a film or glass was left in a dark place for 8 hours. The collected bacteria solution was then diluted and incubated on an agar medium. After incubation, the antibacterial activity value was determined by comparing the counts of colonies formed. The calculation formula is written as R=(Uₜ−U₀)−(Aₜ−U₀)=Uₜ−Aₜ (R: antibacterial activity value, U₀: the mean logarithmic value of the viable bacteria count immediately after inoculation in the untreated product, Uₜ: the mean logarithmic value of the viable bacteria count after 8 hours in the untreated product, Aₜ: the mean logarithmic value of the viable bacteria count after 8 hours in the treated product). The treated product is a coating film obtained by forming a film of a dispersion containing a test target, while the untreated product is a coating film obtained by forming a film of a solution containing a synthetic resin, a polyurethane resin, and a solvent.

### Antibacterial Test 2

With reference to JIS R 1752: 2020, antibacterial testing was performed by the following method on a coating film against Staphylococcus aureus (NBRC 12732) as a representative of gram-positive bacteria and E. coli (NBRC 3972) as a representative of gram-negative bacteria.

A 5×5 cm coating film cleaned by heating in a dry heat sterilizer was inoculated with 0.1 ml of a test bacteria solution. The bacteria solution kept in close contact with a film or glass was irradiated with light (white fluorescent lamp (FL20SSW/18, MITSUBISHI), 500 1x visible light, Sharp Cut Filter Type A (N113, cut off the wavelengths of 400 nm or lower)) and left for eight hours. The collected bacteria solution was then diluted and incubated on an agar medium. After incubation, the antibacterial activity value was determined by comparing the counts of colonies formed. The calculation formula is written as R=(Uₜ−U₀)−(Aₜ−U₀)=Uₜ−Aₜ (R: antibacterial activity value, U₀: the mean logarithmic value of the viable bacteria count immediately after inoculation in the untreated product, Uₜ: the mean logarithmic value of the viable bacteria count after 8 hours in the untreated product, Aₜ: the mean logarithmic value of the viable bacteria count after 8 hours in the treated product). The treated product is a coating film obtained by forming a film of a dispersion containing a test target, while the untreated product is a coating film obtained by forming a film of a solution containing a synthetic resin, a polyurethane resin, and a solvent.

### Antiviral Activity Test 1

With reference to JIS R 1756: 2020, antiviral testing was performed by the following method on a coating film against bacteriophage Qβ (NBRC 20012, host E. coli (NBRC 106373)) and bacteriophage Φ6 (NBRC 105899, host Pseudomonas syringae (NBRC 14084)) as representatives of viruses.

A 5×5 cm coating film was inoculated with a test phage solution. The phage solution kept in close contact with a film or glass was left for four hours in a dark place. The collected phage solution was then diluted and incubated on an agar medium. After incubation, the antiviral activity value was determined by comparing the counts of colonies formed. The calculation formula is written as V_{D}: antiviral activity value (in a dark place): [V_{D}=Log(B_{D})−Log(C_{D})] (D: dark place, B_{D}: the infectivity titer in an untreated product after being left for four hours in a dark place, C_{D}: the infectivity titer in a treated product after being left for four hours in a dark place). The treated product is a coating film obtained by forming a film of a dispersion containing a test target, while the untreated product is a coating film obtained by forming a film of a solution containing a synthetic resin, a polyurethane resin, and a solvent.

### Antiviral Activity Test 2

With reference to JIS R 1756: 2020, antiviral testing was performed by the following method on a coating film against bacteriophage Qβ (NBRC 20012, host E. coli (NBRC 106373)) and bacteriophage Φ6 (NBRC 105899, host Pseudomonas syringae (NBRC 14084)) as representatives of viruses.

A 5×5 cm coating film was inoculated with a test phage solution. The phage solution kept in close contact with a film or glass was irradiated with light (white fluorescent lamp (FL20SSW/18, MITSUBISHI), 500 1x visible light, Sharp Cut Filter Type A (N113, cut off the wavelengths of 400 nm or lower)) and left for four hours. The collected phage solution was then diluted and incubated on an agar medium. After incubation, the antiviral activity value was determined by comparing the counts of colonies formed. The calculation formula is written as V_{D}: antiviral activity value (in a bright place): [V_{L}=Log(B_{L})−Log(C_{L})] (L: bright place, B_{L}: the infectivity titer in an untreated product after being left for four hours in a bright place, C_{L}: the infectivity titer in a treated product after being left for four hours in a bright place). The treated product is a coating film obtained by forming a film of a dispersion containing a test target, while the untreated product is a coating film obtained by forming a film of a solution containing a synthetic resin, a polyurethane resin, and a solvent.

### Antiviral Activity Test 3

With reference to JIS R 1756: 2020, antiviral testing was performed by the following method on an injection-molded product against bacteriophage Qβ (NBRC 20012, host E. coli (NBRC 106373)) as a representative of viruses.

A 5×3 cm injection-molded product was inoculated with a test phage solution. The phage solution kept in close contact with a film or glass was left for 24 hours in a dark place. The collected phage solution was then diluted and incubated on an agar medium. After incubation, the antiviral activity value was determined by comparing the counts of colonies formed. The calculation formula is written as V_{D}: antiviral activity value (in a dark place): [V_{D}=Log(B_{D})−Log(C_{D})] (D: dark place, B_{D}: the infectivity titer in an untreated product after being left for 24 hours in a dark place, C_{D}: the infectivity titer in a treated product after being left for 24 hours in a dark place). The treated product is an injection-molded product including a test target, and the untreated product is an injection-molded product made from the material used in the treated product excluding the test target.

The pigments used in Examples 1 to 12, Evaluation Examples 1 to 14, and Evaluation Comparative Examples 1 to 5 are listed below.

### Iron Phthalocyanine

Iron phthalocyanine used was product name: P-26 (Sanyo Color Works, Ltd.).

### Copper Phthalocyanine

Copper phthalocyanine used was C.I. Pigment Blue 15:3, product name: FASTOGEN BLUE PA5380 (DIC Corporation).

### Quinacridone

Quinacridone used was C.I. Pigment Violet 19, product name: FASTOGEN SUPER RED 500RS (DIC Corporation).

### Isoindolinone

Isoindolinone used was C.I. Pigment Yellow 110, product name: FASTOGEN SUPER YELLOW GRO (DIC Corporation).

Copper compounds used in Examples 1 to 12 are listed below.

### Copper(I) Chloride

Copper(I) chloride available from KANTO CHEMICAL CO., INC. was used.

### Copper(II) Chloride Dihydrate

Copper(II) chloride dihydrate available from KANTO CHEMICAL CO., INC. was used.

### Preparation of Pigment Composition

Based on the formulations in Table 1, pigment compositions of Examples 1 to 12 were prepared by the following formula.

TABLE 1

| | | Ex. | Ex. | Ex. | Ex. | Ex. | Ex. | Ex. | Ex. | Ex. | Ex. | Ex. | Ex. |
|---|---|---|---|---|---|---|---|---|---|---|---|---|---|
| | | 1 | 2 | 3 | 4 | 5 | 6 | 7 | 8 | 9 | 10 | 11 | 12 |
| | Amount of | 1% | 1% | 5% | 5% | 1% | 5% | 5% | 5% | 10% | 20% | 5% | 5% |
| | copper | | | | | | | | | | | | |
| | (wt %) | | | | | | | | | | | | |
| | (copper in | | | | | | | | | | | | |
| | copper | | | | | | | | | | | | |
| | compound/ | | | | | | | | | | | | |
| | pigment) | | | | | | | | | | | | |
| Pigment | Iron | 5.0 | | | | | | | | | | | |
| | phthalocyanine | | | | | | | | | | | | |
| | Copper | | 5.0 | 5.0 | 5.0 | | | | | | | | |
| | phthalocyanine | | | | | | | | | | | | |
| | Quinacridone | | | | | 5.0 | 5.0 | 5.0 | 5.0 | 5.0 | 5.0 | | |
| | Isoindolinone | | | | | | | | | | | 5.0 | 5.0 |
| Copper | Copper(I) | 0.078 | 0.078 | 0.389 | | 0.078 | 0.389 | | | | | 0.389 | |
| compound | chloride | | | | | | | | | | | | |
| | Copper(II) | | | | 0.671 | | | 0.671 | 0.671 | 1.341 | 2.683 | | 0.671 |
| | chloride | | | | | | | | | | | | |
| | dihydrate | | | | | | | | | | | | |
| Heat | 250° C., 3 | None | None | None | None | None | None | None | Applied | None | None | None | None |
| treatment | hours | | | | | | | | | | | | |

Using a 500-mL beaker, the pigment and 15.0 g of ethanol (KANTO CHEMICAL CO., INC.) were mixed and stirred. To this, 300 g of water was added, and the mixture was adjusted to pH 2 using 5% hydrochloric acid. To the resulting solution, a solution prepared by mixing and stirring the copper compound and 30 g of water in a 100-mL beaker and adjusting the mixture to pH 2 using 5% hydrochloric acid was added. After stirring for 10 minutes, the mixture was adjusted to pH 8 using a 5% sodium hydroxide solution. After stirring for another 1 hour, the mixture was filtered, washed, dried, and pulverized to obtain a pigment composition.

The 5% hydrochloric acid was prepared using hydrochloric acid (35 to 37% purity, KANTO CHEMICAL CO., INC.), and the 5% sodium hydroxide solution was prepared using sodium hydroxide (KANTO CHEMICAL CO., INC.).

Based on the formulations in Table 2, coating films of Evaluation Examples 1 to 13 and Evaluation Comparative Examples 1 to 4 were prepared by the following formula.

TABLE 2

| | | Evaluation | Evaluation | Evaluation | Evaluation | Evaluation | Evaluation | Evaluation | Evaluation | Evaluation |
|---|---|---|---|---|---|---|---|---|---|---|
| | | Ex. 1 | Ex. 2 | Ex. 3 | Ex. 4 | Ex. 5 | Ex. 6 | Ex. 7 | Ex. 8 | Ex. 9 |
| Pigment | Ex. 1 | 1.0 | | | | | | | | |
| composition | Ex. 2 | | 1.0 | | | | | | | |
| or pigment | Ex. 3 | | | 1.0 | | | | | | |
| | Ex. 4 | | | | 1.0 | | | | | |
| | Ex. 5 | | | | | 1.0 | | | | |
| | Ex. 6 | | | | | | 1.0 | | | |
| | Ex. 7 | | | | | | | 1.0 | 0.1 | |
| | Ex. 8 | | | | | | | | | 0.1 |
| | Ex. 9 | | | | | | | | | |
| | Ex. 10 | | | | | | | | | |
| | Ex. 11 | | | | | | | | | |
| | Ex. 12 | | | | | | | | | |
| | Iron | | | | | | | | | |
| | phthalocyanine | | | | | | | | | |
| | Copper | | | | | | | | | |
| | phthalocyanine | | | | | | | | | |
| | Quinacridone | | | | | | | | | |
| | Isoindolinone | | | | | | | | | |
| Resin | V343-306SA | 0.2 | 0.2 | 0.2 | 0.2 | 0.2 | 0.2 | 0.2 | 1.7 | 1.7 |
| | IB-D12 | 0.7 | 0.7 | 0.7 | 0.7 | 0.7 | 0.7 | 0.7 | 5.1 | 5.1 |
| Solvent | Toluene | 5.4 | 5.4 | 5.4 | 5.4 | 5.4 | 5.4 | 5.4 | 6.9 | 6.9 |
| | Methyl | 5.4 | 5.4 | 5.4 | 5.4 | 5.4 | 5.4 | 5.4 | 6.9 | 6.9 |
| | ethyl | | | | | | | | | |
| | ketone | | | | | | | | | |

| | | | | | | Evaluation | Evaluation | Evaluation | Evaluation |
|---|---|---|---|---|---|---|---|---|---|
| | | Evaluation | Evaluation | Evaluation | Evaluation | Comp. | Comp. | Comp. | Comp. |
| | | Ex. 10 | Ex. 11 | Ex. 12 | Ex. 13 | Ex. 1 | Ex. 2 | Ex. 3 | Ex. 4 |
| Pigment | Ex. 1 | | | | | | | | |
| composition | Ex. 2 | | | | | | | | |
| or pigment | Ex. 3 | | | | | | | | |
| | Ex. 4 | | | | | | | | |
| | Ex. 5 | | | | | | | | |
| | Ex. 6 | | | | | | | | |
| | Ex. 7 | | | | | | | | |
| | Ex. 8 | | | | | | | | |
| | Ex. 9 | 0.1 | | | | | | | |
| | Ex. 10 | | 0.1 | | | | | | |
| | Ex. 11 | | | 1.0 | | | | | |
| | Ex. 12 | | | | 1.0 | | | | |
| | Iron | | | | | 1.0 | | | |
| | phthalocyanine | | | | | | | | |
| | Copper | | | | | | 1.0 | | |
| | phthalocyanine | | | | | | | | |
| | Quinacridone | | | | | | | 1.0 | |
| | Isoindolinone | | | | | | | | 1.0 |
| Resin | V343-306SA | 1.7 | 1.7 | 0.2 | 0.2 | 0.2 | 0.2 | 0.2 | 0.2 |
| | IB-D12 | 5.1 | 5.1 | 0.7 | 0.7 | 0.7 | 0.7 | 0.7 | 0.7 |
| Solvent | Toluene | 6.9 | 6.9 | 5.4 | 5.4 | 5.4 | 5.4 | 5.4 | 5.4 |
| | Methyl | 6.9 | 6.9 | 5.4 | 5.4 | 5.4 | 5.4 | 5.4 | 5.4 |
| | ethyl | | | | | | | | |
| | ketone | | | | | | | | |

In a 100-mL polyethylene bottle, a pigment composition or a pigment, a synthetic resin (product name: V343-306SA available from DIC Graphics Corporation, solid concentration: 25%, solvent weight ratio: methyl ethyl ketone/ethyl acetate/toluene=35/20/20), a polyurethane resin (product name: SANPRENE IB-D12 available from Sanyo Chemical Industries, Ltd., solid concentration: 30%, solvent weight ratio: methyl ethyl ketone/isopropanol=47/23), a solvent (toluene (available from KANTO CHEMICAL CO., INC.), methyl ethyl ketone (available from KANTO CHEMICAL CO., INC.)), and 40 g of ⅛ inch steel beads were put and shaken with a paint conditioner for 30 minutes to produce a dispersion. The resulting dispersion was applied to a 188 μm-thick PET film using a bar coater No. 6, dried with a dryer, and further dried at 150° C. for 15 minutes to produce coating films of Evaluation Examples 1 to 13 and Evaluation Comparative Examples 1 to 4.

Antibacterial Test 1 and Antiviral Test 1 were performed on the obtained coating films of Evaluation Examples 1 to 13 and Evaluation Comparative Examples 1 to 4. The results are listed in Table 3. Activity values of 0.5 or less were denoted by “poor”. The amount added refers to the percentage of the composition or pigment added to the resin solids.

TABLE 3

| | | | Amount of | | | | | |
|---|---|---|---|---|---|---|---|---|
| | | | copper (wt %) | | | | | |
| | Constituents of | | (copper in | | | | | |
| | composition | | copper | | Activity value | | | |
| Coating | | Copper | compound/ | Amount | Bacteriophage | Bacteriophage | E. | Staphylococcus |
| film | Pigment | compound | pigment) | added | Qβ | Φ6 | coli | aureus |
| Evaluation | Iron | Copper | 1% | 400% | 1.2 | 4.3 | 1.7 | 3.5 |
| Ex. 1 | phthalocyanine | (I) | | | | | | |
| | | chloride | | | | | | |
| Evaluation | Copper | Copper | 1% | 400% | 1.2 | 4.3 | 3.1 | 3.0 |
| Ex. 2 | phthalocyanine | (I) | | | | | | |
| | | chloride | | | | | | |
| Evaluation | Copper | Copper | 5% | 400% | 4.6 | 3.4 | 4.6 | 3.5 |
| Ex. 3 | phthalocyanine | (I) | | | | | | |
| | | chloride | | | | | | |
| Evaluation | Copper | Copper | 5% | 400% | 2.5 | 3.4 | 4.6 | 3.5 |
| Ex. 4 | phthalocyanine | (II) | | | | | | |
| | | chloride | | | | | | |
| Evaluation | Quinacridone | Copper | 1% | 400% | 5.2 | 4.3 | 4.2 | 3.0 |
| Ex. 5 | | (I) | | | | | | |
| | | chloride | | | | | | |
| Evaluation | Quinacridone | Copper | 5% | 400% | 5.2 | 3.4 | 4.6 | 3.5 |
| Ex. 6 | | (I) | | | | | | |
| | | chloride | | | | | | |
| Evaluation | Quinacridone | Copper | 5% | 400% | 3.9 | 3.4 | 3.4 | 3.0 |
| Ex. 7 | | (II) | | | | | | |
| | | chloride | | | | | | |
| Evaluation | Quinacridone | Copper | 5% | 5% | 5.2 | 3.2 | 4.0 | 4.5 |
| Ex. 8 | | (II) | | | | | | |
| | | chloride | | | | | | |
| Evaluation | Quinacridone | Copper | 5% | 5% | 5.4 | 3.8 | 4.5 | 4.0 |
| Ex. 9 | | (II) | | | | | | |
| | | chloride | | | | | | |
| Evaluation | Quinacridone | Copper | 10% | 5% | 3.1 | 1.8 | 2.5 | 2.5 |
| Ex. 10 | | (II) | | | | | | |
| | | chloride | | | | | | |
| Evaluation | Quinacridone | Copper | 20% | 5% | 4.7 | 3.8 | 3.2 | 4.0 |
| Ex. 11 | | (II) | | | | | | |
| | | chloride | | | | | | |
| Evaluation | Isoindolinone | Copper | 5% | 400% | 5.2 | 3.4 | 4.6 | 3.5 |
| Ex. 12 | | (I) | | | | | | |
| | | chloride | | | | | | |
| Evaluation | Isoindolinone | Copper | 5% | 400% | 4.3 | 3.4 | 4.6 | 3.5 |
| Ex. 13 | | (II) | | | | | | |
| | | chloride | | | | | | |
| Evaluation | Iron | — | — | 400% | 1.0 | 4.0 | poor | 2.4 |
| Comp. | phthalocyanine | | | | | | | |
| Ex. 1 | | | | | | | | |
| Evaluation | Copper | — | — | 400% | poor | poor | poor | 0.7 |
| Comp. | phthalocyanine | | | | | | | |
| Ex. 2 | | | | | | | | |
| Evaluation | Quinacridone | — | — | 400% | poor | poor | poor | poor |
| Comp. | | | | | | | | |
| Ex. 3 | | | | | | | | |
| Evaluation | Isoindolinone | — | — | 400% | poor | poor | poor | poor |
| Comp. | | | | | | | | |
| Ex. 4 | | | | | | | | |

According to the results listed in Table 3, the activity values of bacteriophage Qβ, bacteriophage Φ6, E. coli, and Staphylococcus aureus were higher in Evaluation Example 1 than in Evaluation Comparative Example 1, in Evaluation Examples 2 to 4 than in Evaluation Comparative Example 2, in Evaluation Examples 5 to 11 than in Evaluation Comparative Example 3, and in Evaluation Examples 12 and 13 than in Evaluation Comparative Example 4, indicating that the activity was improved by carrying the copper compound.

In Evaluation Example 9, high activity was exhibited even when heat treatment was performed.

Antibacterial Test 2 and Antiviral Test 2 were performed on the obtained coating films of Evaluation Examples 3, 4, 6, 7, 12, and 13 and Evaluation Comparative Examples 2 to 4. The results are listed in Table 4. Activity values of 0.5 or less were denoted by “poor”. The amount added refers to the percentage of the composition or pigment added to the resin solids.

TABLE 4

| | | | Amount of | | | | | |
|---|---|---|---|---|---|---|---|---|
| | | | copper | | | | | |
| | | | (wt %) | | | | | |
| | Constituents of | | (copper in | | | | | |
| | composition | | copper | | Activity value | | | |
| Coating | | Copper | compound/ | Amount | Bacteriophage | Bacteriophage | E. | Staphylococcus |
| film | Pigment | compound | pigment) | added | Qβ | Φ6 | coli | aureus |
| Evaluation | Copper | Copper | 5% | 400% | 5.2 | 3.6 | 4.5 | 3.8 |
| Ex. 3 | phthalocyanine | (I) | | | | | | |
| | | chloride | | | | | | |
| Evaluation | Copper | Copper | 5% | 400% | 2.9 | 3.6 | 4.9 | 3.8 |
| Ex. 4 | phthalocyanine | (II) | | | | | | |
| | | chloride | | | | | | |
| Evaluation | Quinacridone | Copper | 5% | 400% | 5.2 | 3.6 | 4.9 | 2.8 |
| Ex. 6 | | (I) | | | | | | |
| | | chloride | | | | | | |
| Evaluation | Quinacridone | Copper | 5% | 400% | 4.7 | 3.6 | 4.9 | 3.8 |
| Ex. 7 | | (II) | | | | | | |
| | | chloride | | | | | | |
| Evaluation | Isoindolinone | Copper | 5% | 400% | 5.2 | 3.6 | 4.9 | 3.8 |
| Ex. 12 | | (I) | | | | | | |
| | | chloride | | | | | | |
| Evaluation | Isoindolinone | Copper | 5% | 400% | 5.2 | 3.6 | 4.9 | 3.8 |
| Ex. 13 | | (II) | | | | | | |
| | | chloride | | | | | | |
| Evaluation | Copper | — | — | 400% | 1.0 | poor | poor | poor |
| Comp. | phthalocyanine | | | | | | | |
| Ex. 2 | | | | | | | | |
| Evaluation | Quinacridone | — | — | 400% | poor | poor | poor | poor |
| Comp. | | | | | | | | |
| Ex. 3 | | | | | | | | |
| Evaluation | Isoindolinone | — | — | 400% | poor | poor | poor | poor |
| Comp. | | | | | | | | |
| Ex. 4 | | | | | | | | |

According to the results listed in Table 4, the activity values of bacteriophage Qβ, bacteriophage Φ6, E. coli, and Staphylococcus aureus were higher in Evaluation Examples 3 and 4 than in Evaluation Comparative Example 2, in Evaluation Examples 6 and 7 than in Evaluation Comparative Example 3, and in Evaluation Examples 12 and 13 than in Evaluation Comparative Example 4, indicating that the activity was improved by carrying the copper compound.

In Evaluation Example 7, the activity values in Table 4 are higher than those in Table 3, and the differences in activity values especially for bacteriophage Qβ, E. coli, and Staphylococcus aureus are large, indicating that light irradiation is effective.

Based on the formulations in Table 5, injection-molded products of Evaluation Example 14 and Evaluation Comparative Example 5 were produced by the following formula.

TABLE 5

| | | Evaluation | Evaluation |
|---|---|---|---|
| | | Ex. 14 | Comp. Ex. 5 |
| Pigment | Ex. 7 | 5.2 | |
| composition | Quinacridone | | 5.2 |
| or pigment | | | |
| Resin | H-700 | 200 | 200 |
| Others | Magnesium | 5.2 | 5.2 |
| | stearate | | |

A pre-mixture of a pigment and magnesium stearate (product name: SM-1000 available from Sakai Chemical Industry Co., Ltd.) was added to a polypropylene resin (product name: H-700 available from Prime Polymer Co., Ltd.) and mixed well. This mixture was fed into an injection molding machine (model number: PNX60III-5A available from NISSEI PLASTIC INDUSTRIAL CO., LTD.) and molded at 220° C. to obtain injection-molded products of Evaluation Example 14 and Evaluation Comparative Example 5.

Antiviral Test 3 was performed on the obtained injection-molded products of Evaluation Example 14 and Evaluation Comparative Example 5. The results are listed in Table 6. Activity values of 0.5 or less were denoted by “poor”. The amount added refers to the percentage of the composition or pigment added to the resin solids.

TABLE 6

| | | | Amount of | | |
|---|---|---|---|---|---|
| | | | copper | | |
| | | | (wt %) | | Activity |
| | Constituents of | | (copper in | | value |
| Injection- | composition | | copper | | Bacteri- |
| molded | | Copper | compound/ | Amount | ophage |
| product | Pigment | compound | pigment) | added | Qβ |
| Evaluation | Quina- | Copper (II) | 5% | 2.6% | 4.6 |
| Ex. 14 | cridone | chloride | | | |
| Evaluation | Quina- | — | — | 2.6% | poor |
| Comp. Ex. 5 | cridone | | | | |

According to the results in Table 6, the activity value was larger in Evaluation Example 14 than in Evaluation Comparative Example 5, indicating that the activity was improved by carrying the copper compound.

The antibacterial and antiviral agent according to the present invention is water insoluble, has antibacterial or antiviral effect, and has significantly high performance in terms of heat resistance and light fastness, compared with a dye-based antibacterial agent. The antibacterial and antiviral agent according to the present invention has antibacterial or antiviral effect even in the applications such as inks, printed matter, paints, coatings, plastics, fibers, films, and cosmetics, and can be used in a wide range of industrial fields.

What is claimed is:

1. An antibacterial and antiviral agent comprising a pigment composition in which a copper compound is carried on an organic pigment or dye.

2. The antibacterial and antiviral agent according to claim 1, wherein a mass ratio between the organic pigment or dye and the copper compound in the pigment composition is organic pigment:copper compound=99.9:0.1 to 70:30.

3. The antibacterial and antiviral agent according to claim 1, wherein the organic pigment is at least one or more compounds selected from phthalocyanine compounds, quinacridone compounds, and isoindolinone compounds.

4. The antibacterial and antiviral agent according to claim 1, wherein the copper compound is carried in a form of at least one or more compounds selected from Cu₂Cl(OH)₃, CuO, Cu₂O, and CuCl₂·3Cu(OH)₂.

5. An ink, printed matter, paint, coating, plastic, fiber, film, and cosmetic comprising the antibacterial and antiviral agent according to claim 1.
